(19) United States
(12) Patent Application Publication
(10) Pub. No.: US 2021/0041314 A1
(43) Pub. Date: Feb. 11, 2021

(54) USE OF PRESSURE SENSITIVE MATERIAL FOR FORCE FEEDBACK IN A SURGICAL INSTRUMENT

(71) Applicant: TransEnterix Surgical, Inc., Morrisville, NC (US)

(72) Inventors: Damian Brasset, Milano (IT); Matthew Robert Penny, Holly Sprins, NC (US)

(21) Appl. No.: 16/932,661

(22) Filed: Jul. 17, 2020

Related U.S. Application Data
(60) Provisional application No. 62/874,984, filed on Jul. 17, 2019.

(51) Int. Cl. G01L 1/24; A61B 90/92; A61B 1/04; G06T 7/90; A61B 1/00; A61B 34/00; A61B 17/29

(52) U.S. Cl. CPC G01L 1/24; A61B 90/92; A61B 1/04; A61B 2017/00115; A61B 1/00055; A61B 34/76; A61B 17/29; G06T 7/90

(57) ABSTRACT

A surgical instrument has an end effector and a region of stress-responsive colorimetric material on the end effector. The material has a color that changes with increases in pressure applied to tissue by the end effector, and is positioned to be visible to a user or endoscopic camera when in a body cavity engaging tissue. In some applications, image processing techniques are applied to images of the end effector captured during use and can be used to generate feedback to the user if the material is observed to change to a color indicative of a force exceeding a predetermined threshold.

This application claims the benefit of U.S. Provisional Application No. 62/874,984, filed Jul. 17, 2019.

### BACKGROUND

Surgical robotic systems are typically comprised of one or more robotic manipulators and a user interface. The robotic manipulators carry surgical instruments or devices used for the surgical procedure. A typical user interface includes input devices, or handles, manually moveable by the surgeon to control movement of the surgical instruments carried by the robotic manipulators. The surgeon uses the interface to provide inputs into the system and the system processes that information to develop output commands for the robotic manipulator.

In the system illustrated in FIG. 1, a surgeon console 12 has two input devices or handles 17, 18. The input devices are configured to be manipulated by a user to generate signals that are used to command motion of a robotically controlled device in multiple degrees of freedom. In use, the user selectively assigns the two input devices to two of the robotic manipulators 13, 14, 15, allowing surgeon control of two of the surgical instruments 10a, 10b, and 10c disposed at the working site at any given time. To control a third one of the instruments disposed at the working site, one of the two input devices is operatively disengaged from one of the initial two instruments and then operatively paired with the third instrument. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the new haptic interface devices, the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices are configured to be manipulated by a user to generate signals that are processed by the system to generate instructions used to command motion of the manipulators in order to move the instruments in multiple degrees of freedom.

In some surgical systems, sensors are used to determine the forces that are being applied to the patient by the robotic surgical tools during use. Such systems make use of force/torques sensor on a surgical robotic manipulator as a method for determining the haptic information needed to provide force feedback to the surgeon at the user interface.

It can be useful to a surgeon to receive information representing forces between jaws of a surgical instrument and material or tissue being grasped between such jaws. This application describes a new concept for determining such forces, which may be useful in both the context of manually performed surgery as well as in robotic-assisted surgery.

### BRIEF DESCRIPTION OF THE DRAWINGS

FIG. 1 shows an example of a robot-assisted surgical system;

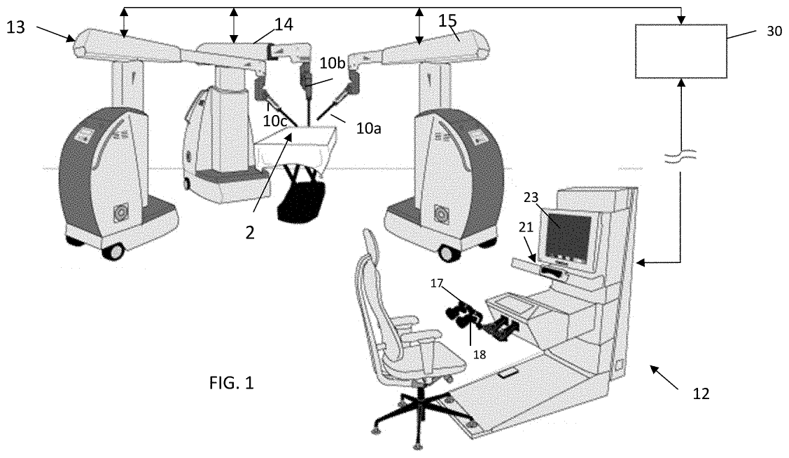

FIG. 2 shows an end effector of a surgical instrument;

FIG. 3 shows a sequence of three images representing an image display from a robot assisted surgical system. Each image shows on the left an instrument using the force sensing principles described herein to communicate forces applied by the jaws of the instrument to tissue.

### DETAILED DESCRIPTION

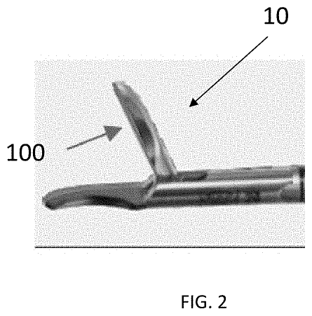

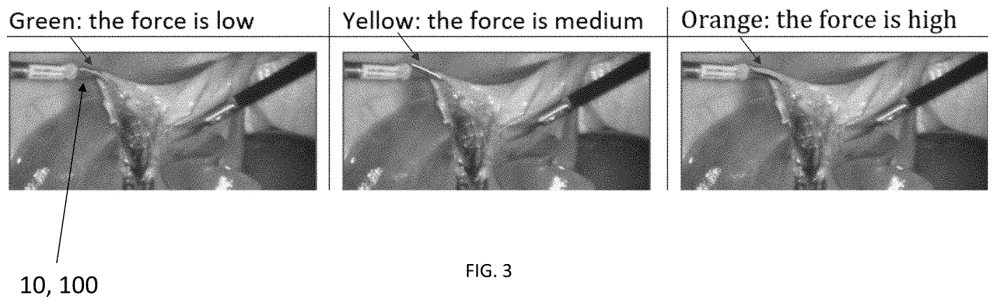

This application describes a surgical instrument that uses materials that are sensitive to force and pressure to convey to a user an indicator of forces applied by the jaws of the instrument to tissue. The material used may be one of the type described in Colorimetric Stress Memory Sensor Based on Disassembly of Gold Nanoparticle Chains, Xiaogang Han et al, Nano Lett. 2014, 14, 5, 2466-2470, Apr. 8, 2014, which is incorporated by reference. The instrument includes an area on which there is a polymer in a location that is visible to the user (or an endoscopic camera positioned in a body cavity to view use of the surgical instrument) and positioned such that the polymer will resolve some or all of the forces applied by the instrument to the tissue. For example, this polymer may be attached to areas of the instrument that will touch tissue during use. For example, wherein the instrument 10 includes jaws as shown in FIG. 2, the polymer 100 is in an area that contacts the jaws when tissue is grasped between the jaws, but that is also sufficiently exposed so that it can be seen by a camera or a user. When force is applied by the jaws, to the tissue, the pressure resolved within the material forces a change in color that is proportional to the force applied, or that may not be proportional but that changes as forces are increased. See FIG. 3. When pressure is released, the material reverts back to its original pigment. The user working at the console vies the procedure on the endoscopic display 23 at the surgeon console and will thus observe the color changes while manipulating the surgical instruments using the input devices 17, 18 at the console.

In addition to the visual feedback given by the color of the polymer to the user, the system's processor may include a memory storing instructions that, when executed, cause the process to receive image data from the endoscopic camera, compare the color of the material as determined from the image data against colors stored in the system's database and correlated with predetermined force thresholds, or ranges of forces stored in categories such as “low, medium, high” forces or other types of camera. Thus the system, with computer vision and an endoscopic camera view will allow a surgical robotic system to detect the color change for each instrument. The system may have stored in a database data correlating forces with colors, and be programmed to alert the user when the force (as determined by recognition of the color using image processing) is determined to be approaching or exceeding a predetermined threshold. This change may be communicated to the user in an additional way, such as using a visual alert such as an overlay generated and displayed on the display, a tactile alert such as vibrational feedback at the instrument handle or other forms of feedback. The user then may alter the force on the user input device handled by the user if s/he feels doing so is warranted. Once the user reduces the force, a second alert may be given notifying the user when the force (as determined by recognition of the color using image processing) is determined to be below the threshold, or beneath the threshold by a predetermined amount.

The alerts given may be one that escalate (e.g. a sound that increases in pitch or frequency as pressure increases; a graphic that changes form, size, color etc as pressure increases; a tactile alert that increases in frequency or intensity as pressure increases).

Another embodiment uses the pressure sensitive material as an integral component in the clevis or shaft of a surgical instrument such that, when the jaws apply force to the tissue, the compressive load on the instrument shaft is resolved, in part, through the pressure sensitive material. This forces a color change proportional to the applied force (or that may not be proportional but that changes as forces are increased) and can be used in the same manner as the first embodiment described above.

We claim:

1. A surgical instrument comprising:
a shaft and an end effector on the shaft;
a region of stress-responsive colorimetric material on the end effector, the material positioned to be visible to a user or endoscopic camera when in a body cavity engaging tissue, the material having a color that changes with increases in pressure applied to tissue by the end effector.

2. A surgical method comprising:
contacting body tissue with a portion of the end effector, causing a first force between the end effector and the tissue, said first force causing a stress-responsive colorimetric material on the end effector to show a first color.

3. The method of claim 2, further including the step of causing a second force between the end effector and the tissue, the second first different than the first force, said second force causing the material to change from the first color to a second color.

4. The method of claim 2, further including:
during the contacting step, capturing an image of the end effector;
applying image processing techniques to the image to identify the first color;
determining whether the first color is indicative of a force exceeding a predetermined threshold; and
if the first color is determined to be indicative of a force exceeding a predetermined threshold, causing a visual, auditory or tactile alert to be communicated to the user.
